(19) United States
(12) Patent Application Publication
(10) Pub. No.: US 2012/0251527 A1
(43) Pub. Date: Oct. 4, 2012

(54) PODOCYTE SPECIFIC ASSAYS AND USES THEREOF

(75) Inventor: Jochen Reiser, Miami, FL (US)

(73) Assignee: University of Miami, Miami, FL (US)

(21) Appl. No.: 13/508,088

(22) PCT Filed: Nov. 8, 2010
(86) PCT No.: PCT/US10/55763

§ 371 Date: Jun. 19, 2012

Related U.S. Application Data
(60) Provisional application No. 61/258,781, filed on Nov. 6, 2009.

(51) Int. Cl. G01N 21/76; C12N 15/63; G01N 33/82; C07K 16/18; A61P 3/10; A61P 13/12; A61P 31/00; A61P 35/00; C12Q 1/68; A61K 39/395

(52) U.S. Cl. 424/130.1; 435/6.1; 435/6.18; 435/320.1; 435/7.21; 435/6.13; 530/350; 435/7.1

(57) ABSTRACT

Compositions are directed to the treatment of kidney diseases in a cell-specific manner. Methods of treating kidney diseases comprise the use of the compositions. Assays for identification of further compounds are provided.

### CROSS REFERENCE TO RELATED APPLICATIONS

This application claims priority to U.S. provisional application No.: 61/258,781, filed Nov. 6, 2009, which is incorporated herein by reference in its entirety.

### FIELD OF THE INVENTION

Embodiments of the invention comprise methods for monitoring molecules associated with kidney diseases. Identification of compositions for the treatment of such diseases and are cell specific.

### BACKGROUND

Nuclear factor of activated T-cells (NFAT) is a general name applied to a family of transcription factors shown to be important in immune response. One or more members of the NFAT family is expressed in most cells of the immune system. NFAT is also involved in the development of cardiac, skeletal muscle, and nervous systems.

The NFAT transcription factor family consists of five members NFATc1, NFATc2, NFATc3, NFATc4, and NFAT5. NFATc1 through NFATc4 are regulated by calcium signaling. Calcium signaling is critical to NFAT activation because calmodulin (CaM), a well known calcium sensor protein, activates the serine/threonine phosphatase calcineurin (CN). Activated CN rapidly dephosphorylates the serine rich region (SRR) and SP-repeats in the amino termini of NFAT proteins resulting in a conformational change that exposes a nuclear localization signal resulting in NFAT nuclear import. Nuclear import of NFAT proteins is opposed by maintenance kinases in the cytoplasm and export kinases in the nucleus. Export kinases, such as PKA and GSK-3β, must be inactivated for NFAT nuclear retention. NFAT proteins have weak DNA binding capacity. Therefore, to effectively bind DNA NFAT proteins must cooperate with other nuclear resident transcription factors generically referred to as NFATn. This feature of NFAT transcription factors enables integration and coincidence detection of calcium signals with other signaling pathways such as ras-MAPK or PKC. Additionally, this signaling integration is involved in tissue specific gene expression during development.

### SUMMARY

This Summary is provided to present a summary of the invention to briefly indicate the nature and substance of the invention. It is submitted with the understanding that it will not be used to interpret or limit the scope or meaning of the claims.

The serine-threonine phosphatase calcineurin regulates the phosphorylation state of synaptopodin—a target for cytosolic cathepsin L. Once synaptopodin is dephosphorylated by calcineurin, 14-3-3 protein can no longer bind to synaptopodin and block cathepsin L cleavage sites in the amino acid sequence of synaptopodin. This leads to degradation of synaptopodin by cytosolic cathepsin L and the development of proteinuria.

In embodiments of the invention, a method of assaying calcineurin activity is via a reporter assay that monitors the activity of NFAT. Phosphorylated NFAT is located in the cytoplasm of podocytes and gets also dephosphorylated once calcineurin is active. Dephosphorylated NFAT travels inside the nucleus and changes transcription.

In a preferred embodiment, an assay system in podocyte cells directly monitors calcineurin/NFAT activity for use in identifying novel agents, diagnostics, disease management and the like.

In another preferred embodiment, screening assays in the screening of chemical compound libraries identify new drugs or as biological assay to screen for disease activity in patient sera.

Other aspects are described infra.

### BRIEF DESCRIPTION OF THE DRAWINGS

FIG. 1 is a schematic representation showing the role of transient receptor potential cation channel 6 (TRPC6) in calcineurin-synpo-NFAT signaling.

FIG. 2 is a schematic representation showing an embodiment of a vector used in the quantitative assessment of NFAT activity by bioluminescence.

FIG. 3 is a graph showing the quantification of NFAT signaling in mouse podocytes with luminescence.

FIGS. 4A and 4B are graphs showing that NFAT is induced by LPS and signals via PKA, calmodulin and calcineurin.

FIGS. 5A and 5B are graphs showing that LPS induction of NFAT is abrogated by dexamethasone.

FIG. 6A is a graph showing LPS induction of NFAT in TPRC6 shRNA is mitigated. Cells were cultured for three weeks under permissive conditions at 37° C. and then treated with LPS (50 μg/ml) for 24 hours and transfected with NFAT response element firefly construct and CMV renilla construct from Promega with LIPOFECTAMINE 2000. 10000 cells per well were lysed and probed with Promega STOP&GLO reagent. All measurements are carried out in octuplicate. FIG. 6B is a graph showing the quantification of NFAT induction caused by LPS in wt and TRPC6 knockdown podocytes.

FIG. 7 is a graph showing that TPRC6 shRNA reduces NFAT induction by FSGS sera. Cells were cultured for two weeks under permissive conditions at 37° C., treated with different sera for one week and transfected with NFAT response element firefly construct and CMV renilla construct from Promega with LIPOFECTAMINE 2000. 10000 cells per well were lysed and probed with Promega STOP&GLO reagent. All measurements are carried out in octuplicate.

FIG. 8 is a graph showing the reduction of baseline NFAT activity in wild type (wt) podocytes that were treated with KN-62, a specific Ca⁺⁺/calmodulin (CaM)-dependent protein kinase inhibitor or with H-89, an inhibitor of cAMP-activated protein kinase A. The use of both inhibitors simultaneously neutralized NFAT activity indicating that baseline NFAT activity is determined by CamKII/PKA.

FIG. 9A is a graph showing NFAT activity is reduced when podocytes are treated with the calcineurin inhibitors cyclosporine A (CsA) and FK506. NFAT activity is decreased in LPS and untreated podocytes. FIG. 9B is the same experiment as in FIG. 9A, but instead of LPS puromycin aminonucleoside (PAN) is used. FIG. 9C is a graph showing that dexamethasone (Dex) also reduces NFAT activity in podocytes under normal conditions and after treatment with LPS. FIG. 9D is a blot showing gene transfer of a podocin-promoter driven plasmid that encodes a TRPC6 pore mutant. The protein has a FLAG tag that can be detected by immunogold analysis in podocyte foot processes (black arrows). FIG. 9E is a graph showing that a proteinuria panel shows no effect of TRPC6 pore mutant expression under normal conditions in podocytes. In contrast, the expression of the TRPC6 pore mutant ameliorated the LPS induced development of proteinuria to a significant degree 24 hours after LPS injection as well as after two injections of LPS at t=48 hours, p*<0.0005.

FIG. 10 is a scan of a photograph showing the TRPC1/5/6 immunogold electron microscopy (EM) in podocyte foot processes which show the locations of these TRPC subunits at the glomerular slit diaphragm.

FIG. 11 is a blot showing human TRPC1 co1Ps. TRPC1 interacts with the slit diaphragm protein podocin and nephrin.

FIG. 12 is a blot showing mouse TRPC5 co1Ps. The TRPC5 interacts with the slit diaphragm protein podocin, nephrin and CD2Ap.

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Embodiments of the present invention relates to discoveries involving methods for monitoring the health of organs such as the kidney. This is especially important in developing new therapies which target specific organs and cells within those organs thereby providing targeted therapies without the drawbacks associated with in vivo systemic drug activities. Compositions for targeting specific molecules and pathways are provided.

Proteinuria is serious sign of kidney impairment that is present in up to 500 million people around the world. Persistent proteinuria can lead to progression of kidney organ loss and is by itself a risk factor for cardiovascular morbidity and mortality. There is no treatment currently available that targets the disease process of proteinuria or the progression in a cell-specific manner.

Without wishing to be bound by theory, the pathways and molecular interactions of the hypothetical proteinuria mechanisms in podocytes are as follows. Phosphorylation of synaptopodin by PKA or CaMKII promotes 14-3-3 binding, which protects synaptopodin against CatL-mediated cleavage, thereby stabilizing synaptopodin steady-state levels. Synaptopodin suppresses IRSp53:Mena-mediated filopodia by blocking the binding of Cdc42 and Mena to IRSp53 and induces stress fibers by competitive blocking the Smurf-1-mediated ubiquitination of RhoA. Synaptopodin also prevents the CatL-mediated degradation of dynamin. Synaptopodin stabilizes the kidney filter by blocking the re-organization of the podocyte actin cytoskeleton into a migratory phenotype. Dephosphorylation of synaptopodin by calcineurin abrogates the interaction with 14-3-3. This renders the CatL cleavage sites of synaptopodin accessible and promotes the degradation of synaptopodin. LPS or various other proximal signals induce the expression of B7-1 and CatL in podocytes, which cause proteinuria through the increased degradation of synaptopodin and dynamin. In parallel, LPS or other proximal signals can also activate Cdc42 and Rac1 though uPAR:b3 integrin signaling, through the loss of synaptopodin-mediated inhibition of Cdc42 signaling or through Nef:Src-mediated activation of Rac1. As a consequence, the podocyte actin cytoskeleton shifts from a stationary to a motile phenotype, thereby causing foot process effacement and proteinuria. CsA and E64 safeguard against proteinuria by stabilizing synaptopodin and dynamin steady-state protein levels in podocytes, FP(4)-Mito by blocking Cdcd42:IRSp53:Mena signaling, cycloRGDfV by blocking uPAR:b3 integrin signaling, NSC23766 by blocking Rac1 and Epleronone by blocking aldosterone signaling.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The terms “determining”, “measuring”, “evaluating”, “detecting”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

As used herein “proteinuria” refers to any amount of protein passing through a podocyte that has suffered podocyte damage or through a podocyte mediated barrier that normally would not allow for any protein passage. In an in vivo system the term “proteinuria” refers to the presence of excessive amounts of serum protein in the urine. Proteinuria is a characteristic symptom of either renal (kidney), urinary, pancreatic distress, nephrotic syndromes (i.e., proteinuria larger than 3.5 grams per day), eclampsia, toxic lesions of kidneys, and it is frequently a symptom of diabetes mellitus. With severe proteinuria general hypoproteinemia can develop and it results in diminished oncotic pressure (ascites, edema, hydrothorax).

As used herein “a patient in need thereof” refers to any patient that is affected with a disorder characterized by proteinuria. In one aspect of the invention “a patient in need thereof refers to any patient that may have, or is at risk of having a disorder characterized by proteinuria.

As used herein, the term “test substance” or “candidate therapeutic agent” or “agent” are used interchangeably herein, and the terms are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. A test substance or agent can be assayed in accordance with the methods of the invention at any stage during clinical trials, during pre-trial testing, or following FDA-approval.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As defined herein, a therapeutically effective amount of a compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

As used herein, the term “reporter gene” refers to a coding sequence attached to heterologous promoter or enhancer elements and whose product may be assayed easily and quantifiably when the construct is introduced into tissues or cells. An example of a “reporter gene” is a nucleic acid encoding a reporter enzyme, i.e., a catalytic product that mediates a reaction of a substrate that produces a detectable signal.

Bioluminescence (BL) is defined as emission of light by living organisms that is well visible in the dark and affects visual behavior of animals (See e.g., Harvey, E. N. (1952). Bioluminescence. New York: Academic Press; Hastings, J. W. (1995). Bioluminescence. In: Cell Physiology (ed. by N. Speralakis). pp. 651-681. New York: Academic Press.; Wilson, T. and Hastings, J. W. (1998). Bioluminescence. Annu Rev Cell Dev Biol 14, 197-230). Bioluminescence does not include so-called ultra-weak light emission, which can be detected in virtually all living structures using sensitive luminometric equipment (Murphy, M. E. and Sies, H. (1990). Meth. Enzymol. 186, 595-610; Radotic, K, Radenovic, C, Jeremic, M. (1998). Gen Physiol Biophys 17, 289-308), and from weak light emission which most probably does not play any ecological role, such as the glowing of bamboo growth cone (Totsune, H., Nakano, M., Inaba, H. (1993). Biochem. Biophys Res Comm. 194, 1025-1029) or emission of light during fertilization of animal eggs (Klebanoff, S. J., Froeder, C. A., Eddy, E. M., Shapiro, B. M. (1979). J. Exp. Med. 149, 938-953; Schomer, B. and Epel, D. (1998). Dev Riot 203, 1-11).

“Optional” or “optionally” means that the subsequently described circumstance may or may not occur, such that the description includes instances where the circumstance occurs and instances where it does not.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

### Compositions

NFAT proteins are direct substrates of calcineurin. Calcineurin is a calmodulin-dependent, cyclosporin A/FK506-sensitive, phosphatase. Calcineurin is activated through its interaction with Ca²⁺ activated calmodulin when intracellular calcium levels are elevated as a result of receptor crosslinking and phospholipase C activation. The activated calcineurin in turn activates NFAT from an inactive cytoplasmic pool. NFAT activation involves protein-protein interaction between calcineurin and NFAT, dephosphorylation of NFAT by calcineurin, conformational change in NFAT resulting from the interaction between calcineurin and NFAT or the dephosphorylation of NFAT and translocation of NFAT to the nucleus. NFAT activation results in induction of NFAT-dependent gene expression of, e.g., cytokine genes.

In a preferred embodiment, a method of assaying calcineurin activity is via a reporter assay that monitors the activity of nuclear factor of activated T cells (NFAT). Phosphorylated NFAT is located in the cytoplasm of podocytes and gets also dephosphorylated once calcineurin is active. Dephosphorylated NFAT travels inside the nucleus and changes transcription. In a preferred embodiment, a reporter system, such as for example, luciferase, in podocytes assays for NFAT activity and thus calcineurin activity. This assay is a very valuable tool allowing the analyze disease activity in patient plasma or the potential of chemical and biological compounds to turn on calcineurin signals.

In one preferred embodiment, the assay provides a read out of disease activity from patient's sera that are about to receive a kidney transplant. Up to 30% of renal transplant recipients can possibly be harmed by recurrent kidney disease. The pathogenesis of this type of disease is unknown and it may be that everything points to a soluble serum factor in the recipient's blood. The NFAT reporter assay herein, was able to read out disease activity by turning on calcineurin/NFAT signals.

In another preferred embodiment, the assay is a screening assay for identification of novel compounds which block the NFAT activation of disease sera. Overall, the described assay constitutes an important contribution to renal disease research and anti-proteinuric therapeutic development. The methods described herein allow for the diagnosis and analysis of disease activity in patient plasma or the potential of chemical and biological compounds to turn on calcineurin signals.

Any aspect of calcineurin-mediated NFAT activation can be evaluated, e.g., protein-protein interaction between calcineurin and NFAT, dephosphorylation of NFAT by calcineurin, recruitment of NFAT to the nucleus in a cell, conformational change in NFAT, or activation of NFAT-dependent gene transcription. These are all examples of NFAT activity and can be measured as discussed herein.

In a preferred embodiment, NFAT activity is assessed quantitatively in a podocyte using a bioluminescent read-out. Aspects of the subject methods include evaluating the activity of a reporter enzyme that is associated with a cell and includes a luminescent enzyme, e.g., luciferase, activity. The term “associated” as used herein includes situations where the reporter enzyme is present inside of the cell, i.e., is present at an intracellular location, as well as situations where the reporter enzyme is present on a surface of a cell, i.e., such that it is located at an intracellular location. A feature of embodiments of the subject methods is that the cell is an intact, living cell, by which is meant that the cell is not dead and the cell's membrane has not been structurally compromised, e.g., via permeabilization, lysis, etc. In preferred embodiments, the cell is a podocyte.

Aspects of the invention include the use of cells that include a luminescent enzyme activity, i.e., the cells include a bioluminescent protein. By luminescent enzyme activity is meant that the cells include an enzyme that converts a substrate to a luminescent product. Any convenient luminescent enzyme may be present in the cell and employed in the subject methods. Representative luminescent enzymes include, but are not limited to: aequorins, luciferases, and the like.

In certain embodiments, the luminescent enzyme activity is a luciferase activity. While the invention is further described below primarily in terms of these embodiments, it is noted that the invention is not so limited, as the guidance provided with respect to luciferase embodiments is readily modified for embodiments that employ other luminescent enzymes. By “luciferase activity” is meant an activity, e.g., enzymatic activity which mediates the transition of a luciferin to a luminescent product. Luciferase activities are enzymes which cause bioluminescence, e.g., by combining their substrate (e.g., luciferyl adenylate) with oxygen. Specific luciferases of interest finding use in certain embodiments include, but are not limited to, those reported in U.S. Pat. Nos. 6,737,245; 6,495,355; 6,265,177; 5,814,465; 5,700,673; 5,674,713; 5,670,356; 5,650,289; 5,641,641; 5,618,722; 5,583,024; 5,352,598; 5,330,906; 5,283,179; 5,229,285; and 5 5,221,623; and 5,219,737.

In certain embodiments, the luciferase activity is a wild-type luciferase or mutant thereof, where specific luciferases of include the following types of wild-type luciferases or mutants thereof: Coleoptera luciferases, e.g., Lampyridae and Elateridae luciferases, including a photinus luciferases, such as luciferases from Photinus aquilonius, Photinus ardens, Photinus collustrans, Photinus consanguineus, Photinus floridanus, Photinus greeni, Photinus ignitus, Photinus indictus, Photinus macdermotti, Photinus marginellus, Photinus obscurellus, Photinus pyralis (common eastern firefly), Photinus sabulosus, and Photinus tanytoxus, where in certain embodiments the luciferase is wild-type Photinus pyralis luciferase or a mutant thereof.

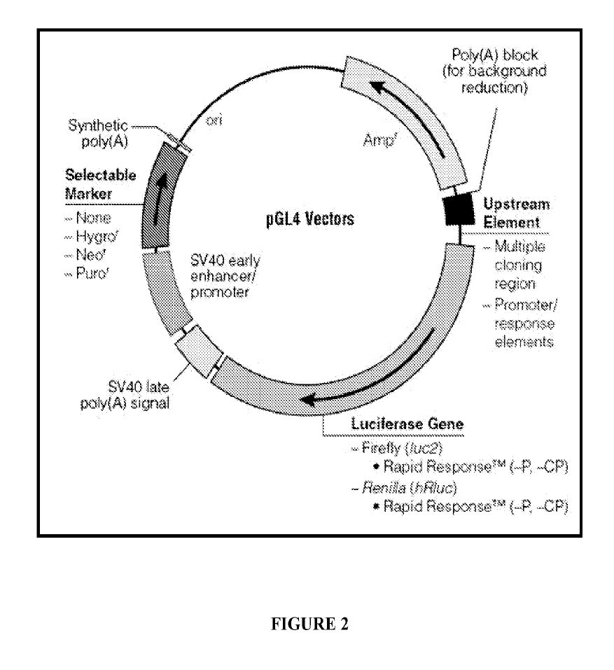

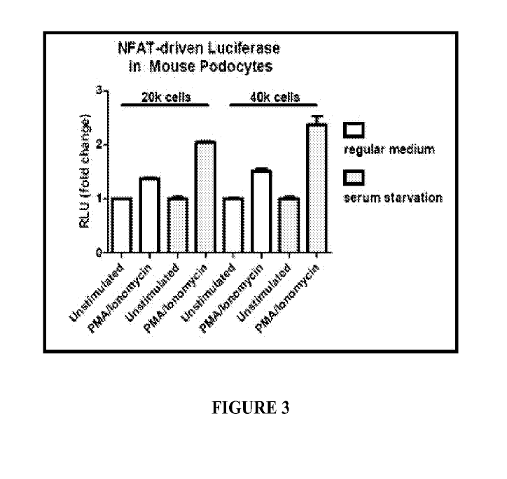

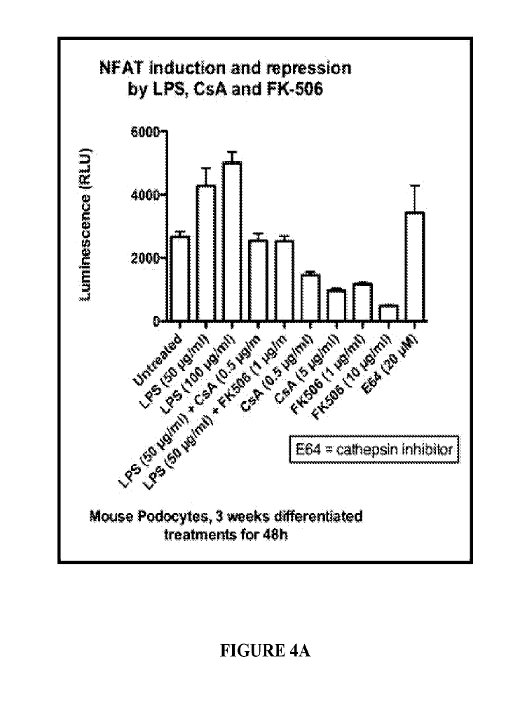

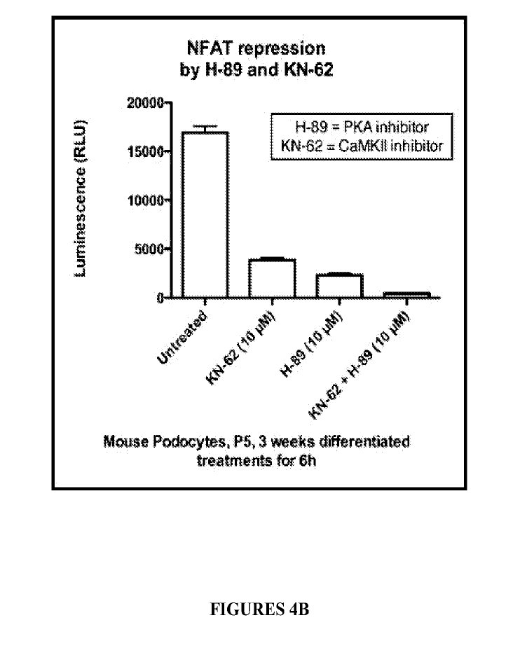

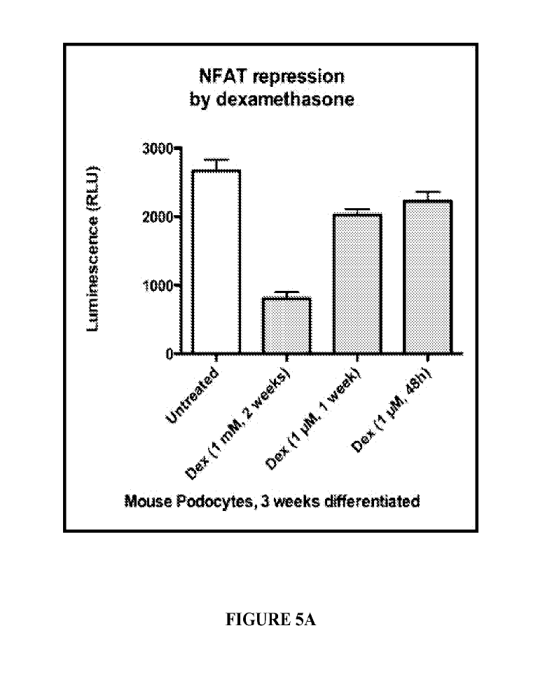

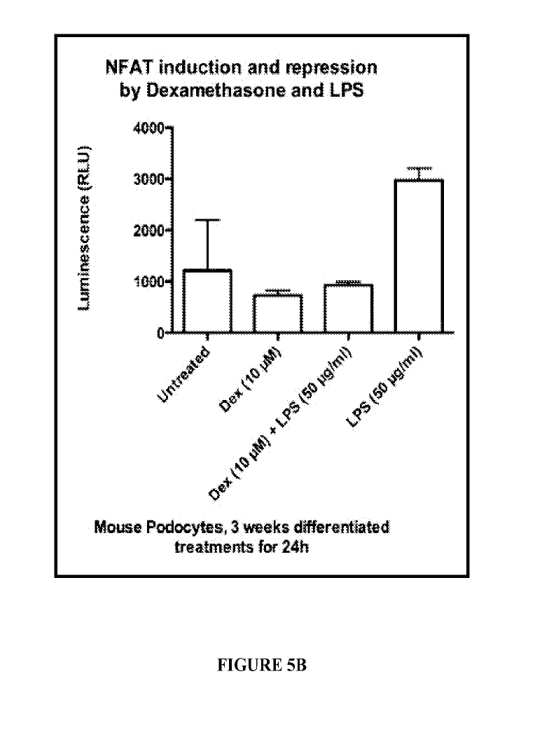

An example of one vector used in the quantitative assessment of NFAT activity by bioluminescence is shown as an example in FIG. 2.

In one preferred embodiment, the vector comprises a luciferase expression cassette which provides for the luciferase activity. In certain embodiments, the luciferase expression cassette is present on a vector that is episomally (i.e., extrachomosomally) maintained in the cell. Expression vectors of interest generally contain a promoter that is recognized by the host organism and is operably linked to the coding sequence for the luciferase coding sequence. Promoters are untranslated sequences located upstream (5′) to the start codon of a structural gene (generally within about 100 to 1000 bp) that control the transcription of particular nucleic acid sequence to which they are operably linked. Such promoters typically fall into two classes, inducible and constitutive. Inducible promoters are promoters that initiate increased levels of transcription from DNA under their control in response to some cellular cue, e.g., the presence or absence of a nutrient, a change in temperature or a developmental or activation signal. Any convenient promoter may be employed.

Transcription from vectors in mammalian cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. Also of interest are promoters for snRNAs, e.g. U1 and U6.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic cell virus. Examples include the SV40 early enhancer/promoter, the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from the 5′ and, occasionally 3′, untranslated regions of eukaryotic or viral DNAs or cDNAs.

In certain embodiments, the expression cassette may be genomically integrated in the target cell, i.e., integrated onto a chromosome of the target cell. A variety of integrating vectors and methodologies for using the same are known in the art and include both site specific and non site-specific integrating systems. For such embodiments, the expression cassette may be placed into a vector that is suitable for use in integrating the expression cassette into the target cell genome, where representative vectors include, but are not limited to: plasmid DNA vectors, retroviral vectors; adeno-associated vectors, adenoviral vectors, double stranded DNA vectors, etc. For example, viral vector delivery vehicles may be employed to integrate an expression cassette into a target cell genome. A variety of viral vector delivery vehicles are known to those of skill in the art and include, but are not limited to: adenovirus, herpesvirus, lentivirus, vaccinia virus and adeno-associated virus (AAV). Transcriptional regulatory elements (promoters, enhancers, terminators, etc.) that find use in genomically-integrated expression cassettes include those noted above for episomal vectors as well as endogenous transcriptional regulatory elements (e.g., as in knock-in gene targeting systems known in the art).

Luciferase expression vectors and methods of using the same, e.g., to transform cells, including cells present in multicellular organisms, are reviewed in U.S. Pat. Nos. 6,737,245; 6,495,355; 6,265,177; 5,814,465; 5,700,673; 5,674,713; 5,670,356; 5,650,289; 5,641,641; 5,618,722; 5,583,024; 5,352,598; 5,330,906; 5,283,179; 5,229,285; and 5 5,221,623; and 5,219,737, the disclosures of which are herein incorporated by reference.

In certain embodiments, the methods are employed in an in vivo bioluminescent imaging protocol, where such protocols include, but are not limited to, those described in U.S. Pat. Nos. 6,939,533; 6,923,951; 6,916,462; 6,908,605; 6,890,515; 6,649,143; 6,495,355; 6,217,847; and 5,650,135. In such embodiments, the methods may include immobilizing a multicellular animal that includes the subject cell(s), administering the reporter molecule or reporter construct and then detecting signal from the animal using whole animal imaging techniques.

In certain embodiments, the methods included introducing the reporter molecule or construct to a multicellular organism at some point prior to signal detection. For example, where the reporter is to be employed for extracellular localization applications, the reporter enzyme may be conjugated to a target moiety for the target cell of interest that is to be localized, and the conjugate administered to the organism, e.g., as reviewed in U.S. Pat. Nos. 6,939,533; 6,923,951; 6,916,462; 6,908,605; 6,890,515; 6,649,143; 6,495,355; 6,217,847; and 5,650,135.

The targeting moiety may be any convenient moiety, where the target moiety is, in many embodiments, properly viewed as an “affinity agent.” In certain embodiments, the affinity agent (i.e., targeting moiety) is a molecule that has a high binding affinity for a target cell, and specifically structure on the target cell. By high binding affinity is meant a binding affinity of at least about 10⁻³ M, such as at least about 10⁻⁶ M or higher, e.g., 10⁻⁹ M or higher. The affinity agent may be any of a variety of different types of molecules, so long as it exhibits the requisite binding affinity for the target cell when immobilized on the surface of a substrate.

### Novel Therapeutics

In preferred embodiments, the methods identify novel therapeutic agents associated with NFAT/calcineurin activities.

The assays can be of an in vitro or in vivo format. In vitro formats of interest include cell-based formats, in which contact occurs e.g., by introducing the substrate in a medium, such as an aqueous medium, in which the cell is present. In yet other embodiments, the assay may be in vivo, in which a multicellular organism that includes the cell is employed. Contact of the targeting vector with the target cell(s) may be accomplished using any convenient protocol. In those embodiments where the target cells are present as part of a multicellular organism, e.g., an animal, the vector or NFAT molecule is conveniently administered to (e.g., injected into, fed to, etc.) the multicellular organism, e.g., a whole animal, where administration may be systemic or localized, e.g., directly to specific tissue(s) and/or organ(s) of the multicellular organism.

Multicellular organisms of interest include, but are not limited to: insect cells, vertebrates, such as avian species, e.g., chickens; mammals, including rodents, e.g., mice, rates; ungulates, e.g., pigs, cows, horses; dogs, cats, primates, e.g., monkeys, apes, humans; and the like. As such, the target cells of interest include, but are not limited to: insects cells, vertebrate cells, particularly avian cells, e.g., chicken cells; mammalian cells, including murine, porcine, ungulate, ovine, equine, rat, dog, cat, monkey, and human cells; and the like.

The target cell comprising the reporter construct is contacted with a test compound and the activity of NFAT is evaluated or assessed by detecting the presence or absence of signal from luciferase substrate, i.e., by screening the cell (either in vitro or in vivo) for the presence of a luciferase mediated luminescent signal. The detected signal is then employed to evaluate the activity of NFAT, since the presence of a detected signal is dependent upon an underlying activity of NFAT.

The luminescent signal may be detected using any convenient luminescent detection device. In certain embodiments, detectors of interest include, but are not limited to: photomultiplier tubes (PMTs), avalanche photodiodes (APDs), charge-coupled devices (CCDs); complementary metal oxide semiconductors (CMOS detectors) and the like. The detector may be present in a signal detection device, e.g., luminometer, which is capable of detecting the signal once or a number of times over a predetermined period, as desired. Data may be collected in this way at frequent intervals, for example once every 10 ms, over the course of a given assay time period.

In certain embodiments, the subject methods are performed in a high throughput (HT) format. In the subject HT embodiments of the subject invention, a plurality of different cells are simultaneously assayed or tested. By simultaneously tested is meant that each of the cells in the plurality are tested at substantially the same time. In general, the number of cells that are tested simultaneously in the subject HT methods ranges from about 10 to 10,000, usually from about 100 to 10,000 and in certain embodiments from about 1000 to 5000. A variety of high throughput screening assays for determining the activity of candidate agent are known in the art and are readily adapted to the present invention, including those described in e.g., Schultz (1998) Bioorg Med Chem Lett 8:2409-2414; Fernandes (1998) Curr Opin Chem Biol 2:597-603; as well as those described in U.S. Pat. No. 6,127,133; the disclosures of which are herein incorporated by reference.

In certain embodiments, the candidate agent is a small molecule or large molecule ligand. By small molecule ligand is meant a ligand ranging in size from about 50 to about 10,000 daltons, usually from about 50 to about 5,000 daltons and more usually from about 100 to about 1000 daltons. By large molecule is meant a ligand ranging in size from about 10,000 daltons or greater in molecular weight.

As indicated above, the present invention finds use in monitoring NFAT/calcineurin activity in a cell (or cells) or patient sample. In certain in vitro embodiments, cells are generated that constitutively express luciferase and harbor a reporter gene construct in which reporter gene expression is controlled by a promoter/enhancer of interest (e.g., a promoter that is turned on in response to a specific cellular cue or one that is indicative of a specific cellular process, e.g., apoptosis). In these embodiments, the cells are cultured under specific user-defined conditions (e.g., in the presence or absence of a cytokine, nutrient and/or candidate therapeutic agent), and monitored for emitted light.

A prototype compound may be believed to have therapeutic activity on the basis of any information available to the artisan. For example, a prototype compound may be believed to have therapeutic activity on the basis of information contained in the Physician's Desk Reference. In addition, by way of non-limiting example, a compound may be believed to have therapeutic activity on the basis of experience of a clinician, structure of the compound, structural activity relationship data, EC₅₀, assay data, IC₅₀ assay data, animal or clinical studies, or any other basis, or combination of such bases.

A therapeutically-active compound is a compound that has therapeutic activity, including for example, the ability of a compound to induce a specified response when administered to a subject or tested in vitro. Therapeutic activity includes treatment of a disease or condition, including both prophylactic and ameliorative treatment. Treatment of a disease or condition can include improvement of a disease or condition by any amount, including prevention, amelioration, and elimination of the disease or condition. Therapeutic activity may be conducted against any disease or condition, including in a preferred embodiment against any disease or disorder associated with proteinuria. In order to determine therapeutic activity any method by which therapeutic activity of a compound may be evaluated can be used. For example, both in vivo and in vitro methods can be used, including for example, clinical evaluation, EC₅₀, and IC₅₀ assays, and dose response curves.

Candidate compounds for use with an assay of the present invention or identified by assays of the present invention as useful pharmacological agents can be pharmacological agents already known in the art or variations thereof or can be compounds previously unknown to have any pharmacological activity. The candidate compounds can be naturally occurring or designed in the laboratory. Candidate compounds can comprise a single diastereomer, more than one diastereomer, or a single enantiomer, or more than one enantiomer.

Candidate compounds can be isolated, from microorganisms, animals or plants, for example, and can be produced recombinantly, or synthesized by chemical methods known in the art. If desired, candidate compounds of the present invention can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomer, or small molecule libraries of compounds and are preferred approaches in the present invention. See Lam, Anticancer Drug Des. 12: 145-167 (1997).

In an embodiment, the present invention provides a method of identifying a candidate compound as a suitable prodrug. A suitable prodrug includes any prodrug that may be identified by the methods of the present invention. Any method apparent to the artisan may be used to identify a candidate compound as a suitable prodrug.

In another aspect, the present invention provides methods of screening candidate compounds for suitability as therapeutic agents. Screening for suitability of therapeutic agents may include assessment of one, some or many criteria relating to the compound that may affect the ability of the compound as a therapeutic agent. Factors such as, for example, efficacy, safety, efficiency, retention, localization, tissue selectivity, degradation, or intracellular persistence may be considered. In an embodiment, a method of screening candidate compounds for suitability as therapeutic agents is provided, where the method comprises providing a candidate compound identified as a suitable prodrug, determining the therapeutic activity of the candidate compound, and determining the intracellular persistence of the candidate compound. Intracellular persistence can be measured by any technique apparent to the skilled artisan, such as for example by radioactive tracer, heavy isotope labeling, or LCMS.

In screening compounds for suitability as therapeutic agents, intracellular persistence of the candidate compound is evaluated. In a preferred embodiment, the agents are evaluated for their ability to modulate the phosphorylation state of NFAT, over a period of time in response to a candidate therapeutic agent. In a preferred embodiment, the phosphorylation state of NFAT in the presence or absence of the candidate therapeutic compound in human tissue is determined. Any technique known to the art worker for determining the phosphorylation state of NFAT may be used in the present invention. See, also, the experimental details in the examples section which follows.

A further aspect of the present invention relates to methods of inhibiting the activity of a condition or disease associated with proteinuria comprising the step of treating a sample or subject believed to have a disease or condition with a prodrug identified by a compound of the invention. Compositions of the invention act as identifiers for prodrugs that have therapeutic activity against a disease or condition. In a preferred aspect, compositions of the invention act as identifiers for drugs that show therapeutic activity against conditions including for example associated with proteinuria.

In one embodiment, a screening assay is a cell-based assay in which the activity of NFAT is measured against an increase or decrease of bioluminescent values in the cells.

In another preferred embodiment, soluble and/or membrane-bound forms of isolated proteins, mutants or biologically active portions thereof, can be used in the assays if desired. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON™ X-100, TRITON™ X-114, THESIT™, Isotridecypoly(ethylene glycol ether)ₙ, 3[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays can also be used and involve preparing a reaction mixture which includes NFAT and bioluminescent molecules and the test compound under conditions and time periods to allow the measurement of the NFAT activity over time, and concentrations of test agents.

In one embodiment, the target product or the test substance is anchored onto a solid phase. The target product/test compound complexes anchored on the solid phase can be detected at the end of the reaction. Preferably, the target product can be anchored onto a solid surface, and the test compound, (which is not anchored), can be labeled, either directly or indirectly, with detectable labels discussed herein.

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

Small molecules may include cyclical carbon or heterocyclic structures and/or aromatic or polyaromatic structures substituted with one or more of the above functional groups. Also of interest as small molecules are structures found among biomolecules, including peptides, saccharides, fatty acids, steroids, purines, pyrimidines, derivatives, structural analogs or combinations thereof. Such compounds may be screened to identify those of interest, where a variety of different screening protocols are known in the art.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e., a compound diversity combinatorial library. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

The whole procedure can be fully automated. For example, sampling of sample materials may be accomplished with a plurality of steps, which include withdrawing a sample from a sample container and delivering at least a portion of the withdrawn sample to test platform. Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

Data and Analysis: The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet.

### Administration of Compositions to Patients

The compositions or agents identified by the methods described herein may be administered to animals including human beings in any suitable formulation. For example, the compositions for modulating protein degradation may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

The compounds can be administered with one or more therapies. The chemotherapeutic agents may be administered under a metronomic regimen. As used herein, “metronomic” therapy refers to the administration of continuous low-doses of a therapeutic agent.

Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

### Formulations

While it is possible for a composition to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical formulations of the present invention, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof Suspending agents such as natural gums, cellulose derivatives or inorganic materials such as silicaceous silicas, and other ingredients such as lanolin, may also be included.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### Regulation of Glomerular Barrier Function by a TRPC6-Synaptopodin Circuit

### Methods

Reagents and Antibodies. The following primary antibodies were used: anti-TRPC6 (antibody 62999 and antibody 12249 Rabbit anti-rat TRPC6 Abcam Plc, Cambridge, UK), Synaptopodin G1D4Progen Biotechnik, Germany, anti-synaptopodin G1 and NT (Mundel et al JCB 1997), Anti-Desmin D33 DAKO, Denmark, anti-alpha-actinin-431, FITC-conjugated phalloidin (Sigma).

Animal studies. (1) Passive Heyman Nephritis: Male Wistar rats with an initial weight of approximately 150 g were housed in a light and temperature-controlled room with ad libitum access to drinking water and standard pelleted chow. PHN was induced by a single injection of sheep-anti-Fx1a antibody that was raised as described previously (Shankland, S. J., Pippin, J. W., Reiser, J. & Mundel, P. Kidney Int 72, 26-36 (2007)). After 18 and 33 weeks, respectively, the animals were housed in metabolic cages enabling collection of 24 h urine samples. Subsequently, the animals were anesthetized and sacrificed by cervical dislocation. Immediately thereafter, kidney was sampled and frozen in liquid nitrogen until further processing. The animal ethics board of the Radboud University Nijmegen approved all animal studies. (2) Lipopolysaccharide (LPS) mouse model: The LPS model was used as described before (Reiser, J., et al. Induction of B7-1 in podocytes is associated with nephrotic syndrome. J Clin Invest 113, 1390-1397 (2004)).

Immunohistochemistry. Kidney tissue immunofluorescence staining was performed on 2 μm kidney cryosections. After fixation by application of 2% paraformaldehyde with 4% sucrose in phosphate buffered saline (PBS), permeabilization in 0.3% Triton-X100 in PBS and blocking in 2% BSA with 2% FCS and 0.2% fish gelatin in PBS, the sections were incubated with the respective primary antibodies and, subsequently, Alexa-conjugated secondary antibodies. Stained sections were embedded in Vectashield mounting medium H1000 (Vector Laboratories Inc., Burlingame, Calif.). Images were made on a Zeiss microscope. Glomeruli were scored from 0 to 4 based on the positive staining in the glomerulus (negative=0, 1-25% positive=1, 26-50% positive=2, 51-75% positive=3 and 76-100% positive=4). All scoring was performed independently by 2 investigators, which were blinded for the specific treatment groups and scored 40-60 glomeruli per animal. Immunocytochemical analysis of cultured podocytes was performed as described previously (Mundel, P., Reiser, J., Záñiga Mejía Borja, A., Pavenstädt, H., et al. Exp Cell Res 236, 248-258 (1997)).

Immunoblotting. SDS-PAGE and Western blotting were done as described before with the modification that Invitrogen's blot module (XCell Sure-Lock Tank), gels (4-12% NuPAGE Bis-Tris), running (MES or MOPS) and transfer buffers were used.

Cell culture and transient transfection. Mouse podocytes were cultured as described previously (Mundel, P., Reiser, J., et al. Exp Cell Res 236, 248-258 (1997); Shankland, S. J., Pippin, J. W., Reiser, J. & Mundel, P. Kidney Int 72, 26-36 (2007)). HEK293 cells were maintained and transfected as previously reported (Reiser, J., et al. Nat Genet 37, 739-744 (2005)).

Co-immunoprecipitation and immunoblot analysis. Briefly, cells were lysed in 50 mM Tris-Cl, pH 7.6, 150 mM NaCl, 1% Triton X-100, 1% sodium deoxycholate, 2 mM EDTA, 1 mM PMSF, and protease inhibitor mixture (Sigma). Lysates were cleared by centrifugation and the resulting extracts (500 μg of protein) were incubated in the presence of anti-synaptopodin, or IgG (1-2 μg), for 4 h at 4° C. 20 μl of protein A/G agarose (Santa Cruz Biotechnology) was added to the lysates and incubated for 12 h. Pellets were washed, boiled for 5 min in SDS sample buffer, and proteins were separated by SDS-PAGE on 10% gels, and transferred to filters. Cell extracted protein (50-100 μg) was used as control in each experiment. Blots were blocked, washed, incubated with the primary antibody overnight at 4° C., washed again, and the membrane was incubated with horseradish peroxidase-conjugated secondary antibody for 1 h at room temperature. The proteins were visualized using a chemiluminescent substrate (SuperSignal West Pico, Pierce Biotechnology). A dilute sample of cell lysate was used to determine electrophoretic mobility of the interacting proteins, and is labeled as “Input” in figures.

Cell-surface biotinylation assays. These were carried out as described in detail previously (Kim, E. Y., et al., Mol Pharmacol 75, 466-477 (2009); Kim, E. Y., et al. Am J Physiol Renal Physiol 295, F235-F246 (2008)). Briefly, HEK293T cells transiently expressing synaptopodin and TRPC6, or differentiated cells from different podocyte cell lines, were treated with a membrane impermeable biotinylation reagent, sulfo-N-hydroxy-succinimidobiotin (Pierce Biotechnology, Rockford, Ill.) (1 mg/ml in PBS buffer) for 1 h. The reaction was stopped, cells were lysed, and biotinylated proteins from the cell surface were recovered from lysates by incubation with immobilized streptavidin-agarose beads (Pierce Biotechnology). A sample of the initial cell lysate was retained for analysis of total proteins. These samples were separated on SDS-PAGE, and proteins were quantified by immunoblot analysis followed by densitometry using ImageJ software (National Institutes of Health). These experiments were repeated three times.

Isolation and processing of glomeruli. Glomeruli were isolated from kidneys of 8-12 weeks old LPS- and PBS-treated (control) mice using a sequential sieve technique with mesh sizes of 180, 100, and 71 μm. The fraction collected from the 71-μm sieve was maintained for soup/pellet fractionation. Isolated glomeruli were homogenized in buffer containing 20 mM HEPES pH 7.5, 100 mM NaCl, 1 mM MgCl₂, 1 mM PMSF, protease inhibitors (Roche), calpain inhibitor (Calbiochem), and E-64d (Calbiochem) using Dounce homogenizer. Subsequently, cytosol was centrifuged for 10 min at 4,600 g. Proteins were solubilized by 1% Triton X-100, 1 hour at 4° C., before it was spun at 70,000 g for 1 hour.

In vivo gene delivery. A TRPC6 pore mutant plasmid (Hofmann, T., et al., Proc Natl Acad Sci U S A 99, 7461-7466 (2002)) was introduced into mice (n>10, each construct) using the TransIT in vivo gene delivery system (Mirus) as described previously (Möller, C. C., et al. J Am Soc Nephrol 18, 29-36 (2007)).

Mouse phenotyping. Freshly harvested kidneys were fixed in 4% PFA (Electron Microscopy Sciences) solution. They were then embedded in paraffin and 2 micron sections cut and stained with hematoxylin and eosin (H&E), periodic acid-Schiff (PAS) reagent or methenamine-silver stain. The sections were examined in a blinded manner and scored for glomerular and other renal changes. Glomerular lesion scores were assigned on a 4 point scale based on the number of glomeruli involved and the severity of the lesions (1, 1 score; 2, 2-3 scores; 3, 3 and above scores; 4, 3 and above with confluency). Overall lesion scores included focal hypercellularity, glomerulosclerosis (FSGS), crescent formation, epithelial cell reactivity (i.e., podocyte enlargement and/or hyperplasia which might cause bridging to Bowman's capsule) and apoptosis. Thirty glomeruli in each kidney were examined. Urine microalbumin was assessed by the densitometric analysis of the Bis-Tris gels loaded with the standard BSA (Bio-Rad Laboratories) and the urine samples. The urinary creatinine measurement was carried out using a colorimetric end-point assay with a commercial kit (Cayman Chemical).

Electrophysiological analysis of TRPC6 currents. HM1 cells (human embryonic kidney cells stably transfected with the M1 muscarinic receptor) were transiently transfected with wild-type (WT) TRPC6 and synaptopodin constructs. Electrophysiological recordings were conducted between 36-48 h after transfection. All patch-clamp experiments were performed at room temperature (20-25° C.). Whole-cell currents were recorded using an Axopatch 200B amplifier. Patch electrodes were pulled from borosilicate solutions. Series resistance (Rs) was compensated up to 90% to reduce series resistance errors to <5 mV. Cells in which Rs was >10 MΩ were discarded. For whole cell currents recordings, voltage stimuli lasting 250 ms were delivered at 1- to 5-s intervals, with voltage ramps ranging from −120 to +100 mV. The holding potential was 0 mV. A fast perfusion system was used to exchange extracellular solutions, with complete solution exchange achieved in ˜1 to 3 s. The internal pipette solution for whole cell current recordings contains (in mM) 145 Cs-methanesulfonate, 10 NaCl, 2 Mg-ATP, 0.2 Na-GTP, 1 EGTA, 0.38 CaCl₂ and 10 HEPES buffer (pH 7.2 adjusted with CsOH). Free calcium concentration in the pipette solution was 100 nM as calculated by MaxChelator. The standard extracellular Tyrode's solution for whole cell current recording contains (mM): 145 NaCl, 5 KCl, 2 CaCl₂, 1 MgCl₂, 10 HEPES and 10 glucose; pH was adjusted to 7.4 with NaOH, and osmolarity was adjusted to ˜300 mOsm. N-methyl D-glucamine (NMDG) solution was prepared using (mM) 145 NMDG-Cl, 10 HEPES, and 10 glucose (pH 7.4). For some experiments, 10 mM Ca² was prepared in NMDG solution. The concentration of NMDG was reduced accordingly in order to keep the identical osmolarity.

Podocyte-NFAT reporter assays: A podocyte cell line that stably expressed pGL4.30 reporter plasmid (Promega) was generated. pGL4.30 includes the luc2P firefly luciferase gene under the control of the NFAT response element. For LPS treatments both wt and stable TRPC6 knockdown podocyte cell line were transiently transfected with the pGL4.30 and pGL4.74 reporter plasmids (Promega). pGL4.74 includes the hRluc Renilla luciferase gene under the control of the constitutive HSV-TK promoter, serving as an internal control for normalization of the results. Cells were seeded in 96 well plates in standardized cell number and differentiated for three weeks prior to transfection and treatments. Stable cell lines were probed with BRIGHT-GLO Luciferase Assay System (Promega), transiently transfected cells were assayed with DUAL-GLO Luciferase Assay System (Promega). Luminescence was measured on a Molecular Devices luminescence microplate reader SpectraMax L. All experiments were carried out in octuplicate per condition and repeated in three independent experiments.

Podocytes rescue experiments TRPC6 knock down (TPRC6 KD) and scrambled podocytes were grown under growth-restrictive conditions for 13-15 days before treatment. Cells were serum-starved in RPMI medium containing 0.2% FBS to avoid serum effects and were treated with the following drugs as indicated: 10 μM forskolin (Sigma), 1 mg/ml Cyclosporin A (CsA, Sigma), 20 μM Cathepsin L inhibitor (Calbiochem). Whole cell extracts were isolated using RIPA buffer. Western blot analysis was performed according to standard procedures using rabbit anti-TRPC6 antibody (Abcam), rabbit N-terminal synaptopodin (NT) and mouse anti-GAPDH as a loading control.

Statistical analysis. Statistical analysis was performed by Student's t-test with the level of significance set at P<0.05. Data are reported as mean values +/− standard error of the means.

Results:

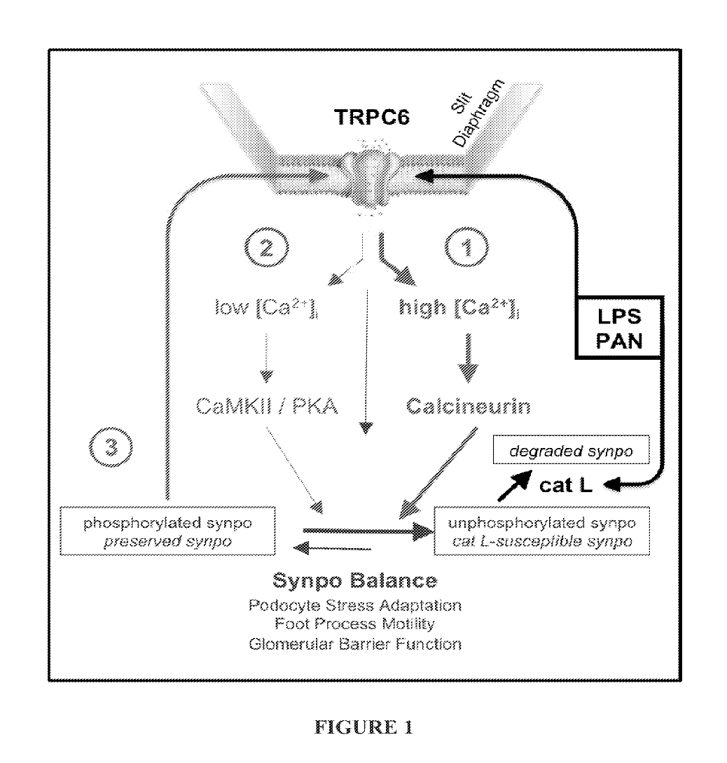

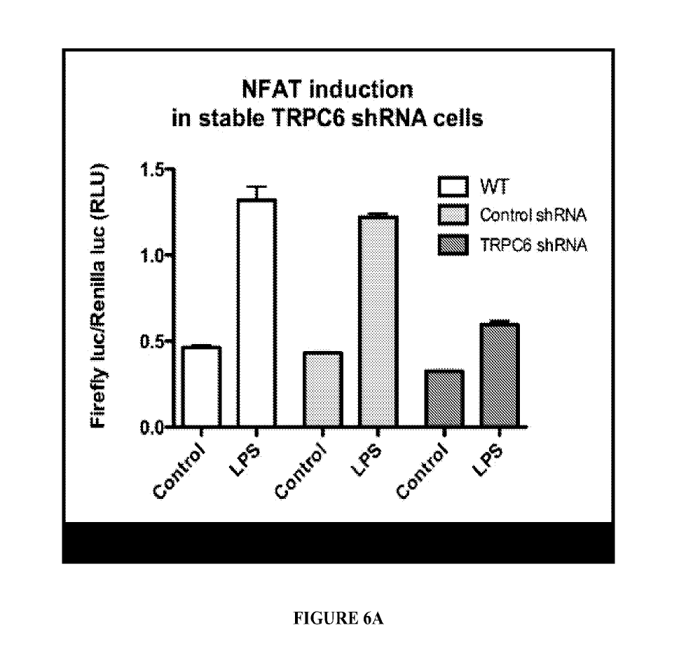

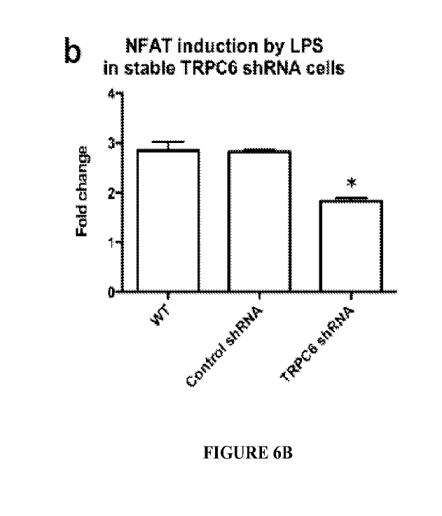

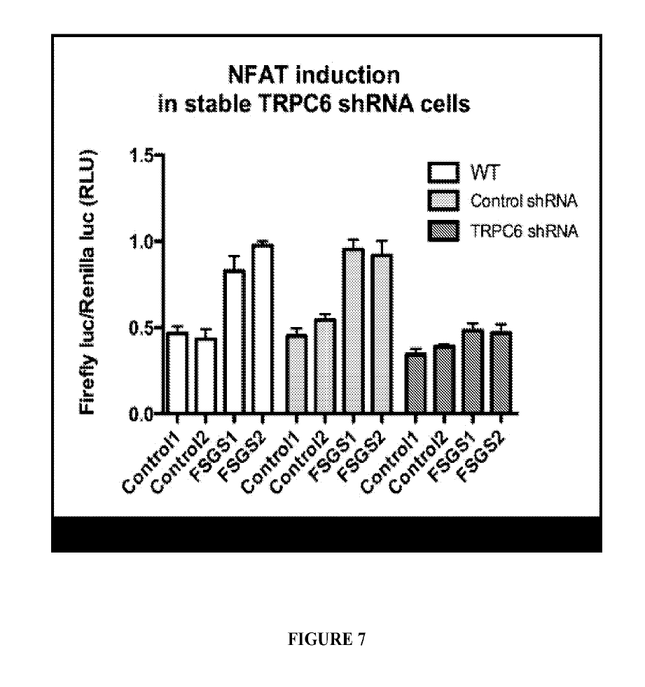

TRPC6 in podocytes is required for proper stimulated Ca²⁺ influx: TRPC6 mutations can lead to increased channel function and cause familial focal segmental glomerulosclerosis (FSGS). Induction of wt TRPC6 is associated with acquired glomerular disease including Membranous Nephropathy. To better define the role of Ca²⁺ channeled through podocyte TRPC6 in a physiological cellular context, podocyte cell lines that express normal and reduced levels of TRPC6 using stable expression of TRPC6 shRNA, were analyzed. TRPC6 expression was reduced on mRNA and protein level. The functional impairment of TRPC6 knockdown podocytes is demonstrated by reduced OAG-stimulated Ca²⁺ influx into cells that were loaded with the Ca²⁺ indicator Fura-2. As additional functional evaluation of the Ca⁺ signaling pathway in normal and TRPC6 knockdown podocytes, the Nuclear factor of activated T-cells (NFAT) activity assay was used, that can assay the cellular downstream effects of Ca²⁺ along the calmodulin/calcineurin pathway. After transient transfection of podocytes with a NFAT-responsive luciferase reporter plasmid (FIGS. 6A, 6B). TRPC6 channel activity enhances Nuclear factor of activated T-cells (NFAT) signaling in cardiomyocytes as well as in cultured HEK293 cells that express wt or mutated TRPC610. Podocytes had a low baseline activity of NFAT that was strongly increased after pretreatment of the cells with lipopolysaccharide (LPS) (FIG. 6A) which is in line with increased Ca⁺ transport due to augmented TRPC6 channel expression. In contrast, the knockdown of TRPC6 led to a decreased activation of NFAT after LPS stimulus (FIG. 1B). In summary, TRPC6 is required for proper amounts of podocyte Ca²⁺ and facilitates signals along the calmodulin-calcineurin pathway.

Synaptopodin interacts with TRPC6 to regulate its membrane localization and channel activity: Since TRPC6 mediated Ca²⁺ influx stimulated calcineurin activity in podocytes, synaptopodin that has recently been shown to be a substrate for calcineurin and that is important in maintaining podocyte RhoA levels ensuring proper podocyte functioning, was examined. Co-immunoprecipitation studies in HEK293 cells that were co-transfected with various synaptopodin constructs as well as TRPC6 were conducted. The results showed an interaction of TRPC6 with synaptopodin long (kidney form) and short (brain form) but not with the alternatively expressed isoform synaptopodin T that was originally identified as developmental backup protein in synaptopodin deficient mice. This interaction of TRPC6 with synaptopodin was also confirmed by endogenous immunoprecipitation in cultured podocytes. Since synaptopodin was found along actin stress fibers as well as in the cortical actin web, it was queried whether synaptopodin could be involved in trafficking or tethering TRPC6 to the plasma membrane of podocytes. Surface biotinylation studies were performed in TRPC6 and synaptopodin co-transfected HEK293 cells as well as in wt podocytes and in podocytes that have stably downregulated synaptopodin expression. In both cellular systems, a larger amount of TRPC6 at the plasma membrane was found when synaptopodin was present demonstrating that synaptopodin is required for proper localization of TRPC6 at the plasma membrane. Since a higher plasma membrane expression of TRPC6 should possibly result in elevated Ca²⁺ influx, electrophysiological studies were performed and Ca²⁺ influx was recorded in carbachol stimulated HEK293 cells that express TRPC6 alone or in combination with synaptopodin, a synaptopodin phosphomimetic mutant as well as with α-actinin. It was found that both expressed synaptopodin proteins but not α-actinin significantly augment TRPC6 mediated Ca⁺ influx.

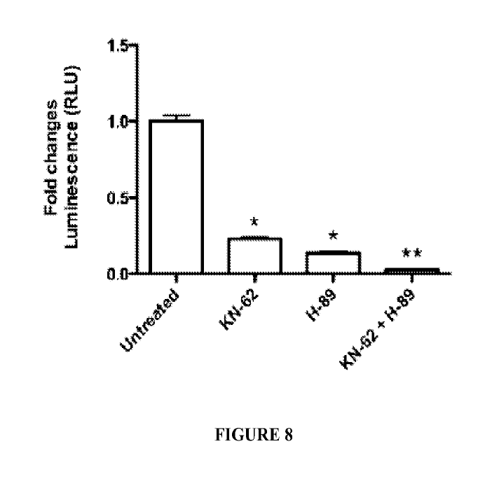

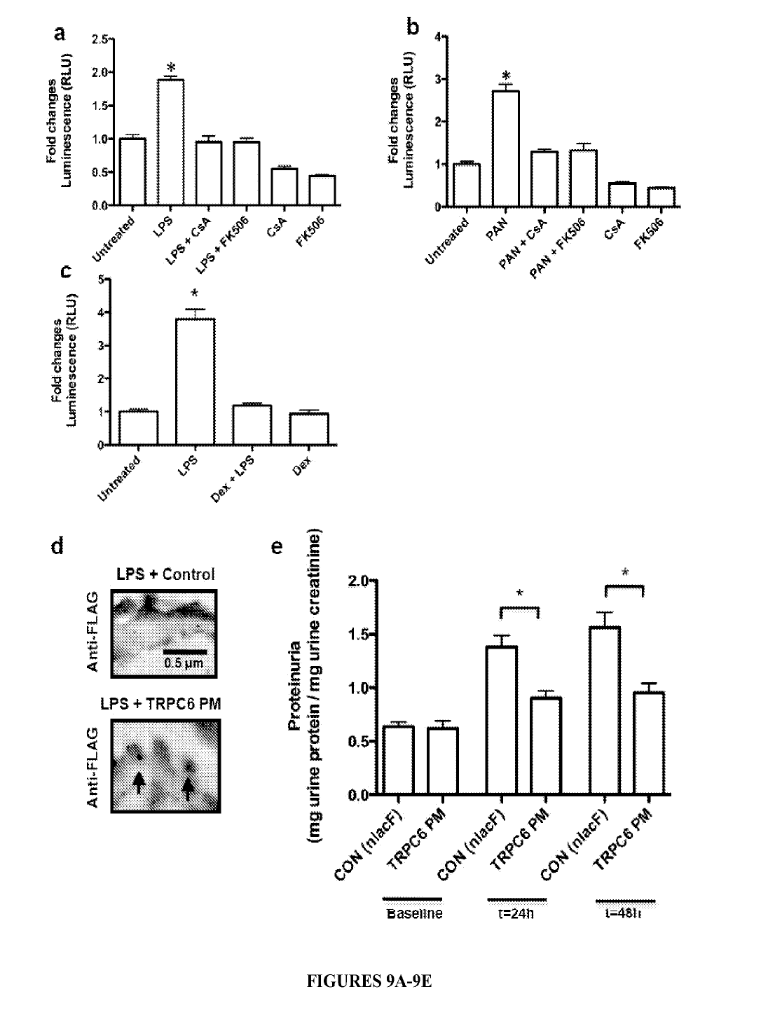

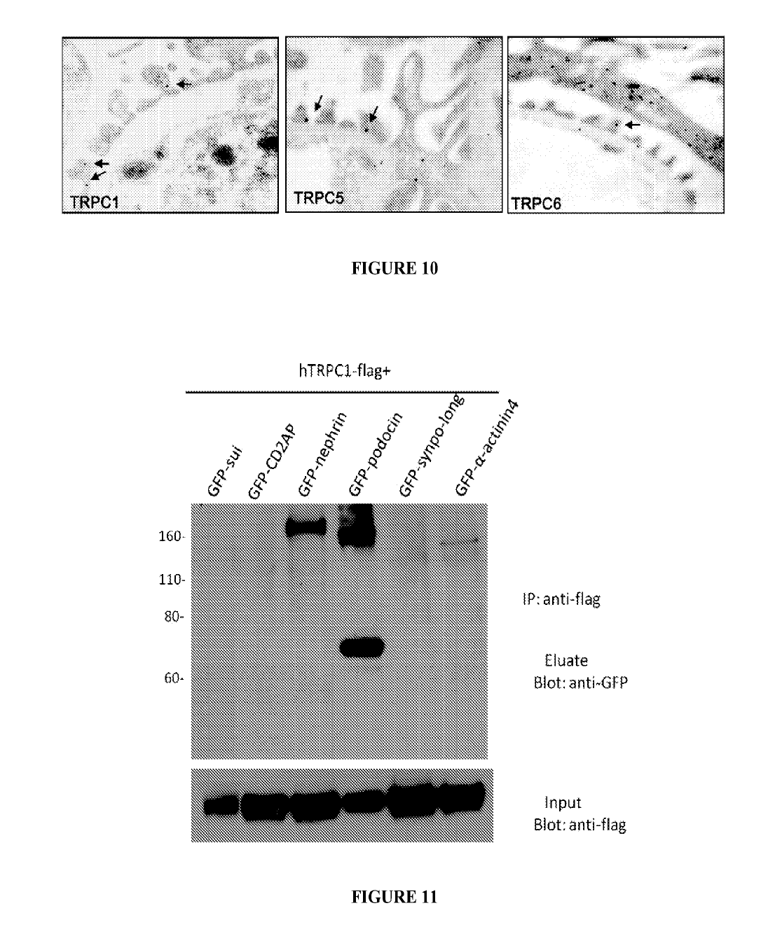

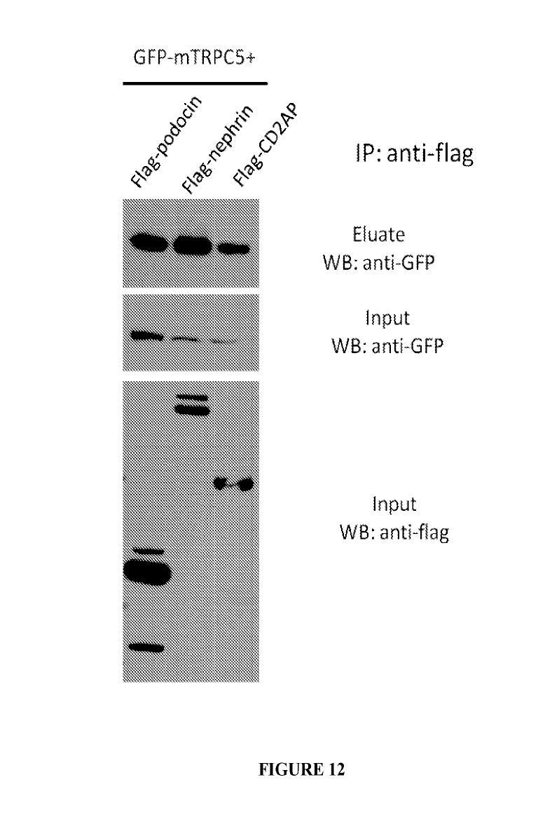

TRPC6 is required to maintain physiological synaptopodin levels and a functioning podocyte cytoskeleton: Since synaptopodin is required for TRPC6 localization to the podocyte plasma membrane and TRPC6 is required for physiological Ca²⁺ levels in the podocyte that could affect Ca²⁺ sensitive enzyme systems such as PKA and calcineurin that regulate synaptopodin stability, F-actin organization was examined as well as the expression of synaptopodin in TRPC6 knockdown podocytes. Interestingly the normal parallel stress fiber pattern in wt and control shRNA expressing podocytes changed to a reduced and a diffusely distributed actin within the cytoplasm of TRPC6 knockdown podocytes with prominent submembranous concentration. The appearance was reminiscent of a F-actin pattern in podocytes after LPS7 or Puromycin-aminonucleoside treatment but also after overexpression of TRPC65. When synaptopodin distribution was analyzed, a reduction in overall stress-fiber associated synaptopodin and a more speckled distribution was found. Overall, the F-actin/synaptopodin pattern could represent a compensatory response to shift the remaining small amounts of TRPC6 to the plasma membrane to channel enough Ca²⁺ for PKA activity in an attempt to phosphorylate and stabilize synaptopodin. The loss of stress fibers in TRPC6 downregulated podocytes is best explained by synaptopodin degradation. In the absence of physiological cellular Ca²⁺ levels synaptopodin protein stability is reduced because of reduced PKA mediated serine/threonine phosphorylation. In fact, when rescue experiments of TRPC6 knockdown podocytes were performed with forskolin, an activator of PKA, synaptopodin expression increased, evidencing that in the absence of TRPC6, PKA activity is reduced. After stimulation of PKA with forskolin an increased PKA mediated serine threonine phosphorylation of synaptopodin stabilizes its expression even in the absence of TRPC6 as PKA lies downstream of it. A similar increase in synaptopodin can be observed after inhibition of synaptopodin degradation using the cathepsin L inhibitor Z-FF-FMK. The effects of the calcineurin inhibitor cyclosporin A (CsA) on synaptopodin expression were studied. Low synaptopodin expression in TRPC6 knockdown podocytes was increased but to a lower degree than after treatment with Forskolin or cathepsin L inhibitor. These studies were also corroborated in wt podocytes showing that inhibition of PKA or Ca²⁺/Calmodulin-dependent protein kinase II (CaMKII) leads to a significant reduction of NFAT activity arguing for a high baseline activity of PKA and CaMKII that are reduced in TRPC6 knockdown podocytes (FIG. 8). Podocyte motility was studied next because it requires the proper organization and function of the F-actin cytoskeleton. A wound healing (directed motility) and Boyden chamber (random motility) assays, were performed (Wei, C., et al. Nat Med 14, 55-63 (2008)). Both were significant for a reduced motility in podocytes that have stable reduction of TRPC6 but not in podocytes that express the scramble shRNA or in control wt cells indicating that physiological podocyte Ca²⁺ levels are required for proper cytoskeletal organization and function.

TRPC6 induction is associated with synaptopodin loss in vivo: Having identified a unique functional interaction between TRPC6, PKA, calcineurin and synaptopodin, the Passive Heymann Nephritis rat (PHN) glomerular disease model that is characterized by a strong increase in wt TRPC6 expression, was analyzed. A strong segmental increase was noted in TRPC6 glomerular expression in rats that have developed well established Passive Heymann Nephritis after 18 weeks and after 33 weeks of disease induction compared to age-matched control rats. The induction of TRPC6 expression correlated well with the degree of albuminuria (r=0.816). Interestingly, it was noted synaptopodin loss in areas that were characterized by TRPC6 induction evidencing that increased TRPC6 has led to a degradation of synaptopodin. To study if other proteins in podocytes were also associated with a decreased expression in areas of TRPC6 induction in this model of Membranous Nephropathy, a double immunofluorescent labeling of TRPC6 with the podocyte damage marker desmin was performed. Desmin expression was unchanged 18 and 33 weeks after disease induction and not affected by the increase in segmental TRPC6 expression. To better define if the calcineurin pathway was involved in the partial degradation of synaptopodin, cultured podocytes were exposed to proteinuria injury stimuli such as LPS and PAN in the presence or absence of various calcineurin inhibitors as well as steroids (FIGS. 9A-9C). It was found that calcineurin inhibitors as well as by steroids reduce calcineurin activity arguing that podocyte injury signals commonly activate calcineurin in podocytes leading to dephosphorylation of synaptopodin and its cathepsin L mediated degradation.

Maintaining TRPC6 balance stabilizes synaptopodin and ameliorates proteinuria: To evaluate the potential value of neutralization of overly expressed TRPC6, two approaches were used: 1) in vivo delivery of a siRNA construct that downregulates TRPC6 expression in podocytes and 2) gene delivery of a podocyte-specific TRPC6 dominant-negative pore mutant. First, a siRNA construct was delivered, which was coupled to an anti-podocyte antibody and thus provided a podocyte-specific delivery technique (shamporter). This system uses the antigenic site of an anti-podocyte antibody that is coupled to TRPC6 siRNA. Using this approach, TRPC6 was effectively downregulated expression in podocytes during Passive Heyman Nephritis and consequently also normalized the levels of synaptopodin expression in the glomerulus. This treatment was associated with a significant reduction of proteinuria in Passive Heymann Nephritis Rats 4 days after TRPC6 siRNA injection. One week after siRNA delivery, there was a prominent reduction of proteinuria in Passive Heymann Nephritis rats. The effects of a plasmid injection that encodes a Flag-tagged TRPC6 pore mutant driven by the podocyte-specific podocin promoter were analyzed (FIGS. 9D-9E). LPS-treated mice that concomitantly received the plasmid encoding for the TRPC6 pore mutant displayed a significant amelioration in proteinuria development (FIG. 9E). Podocyte-specific expression of the TRPC6 pore mutant was detected in podocyte foot processes in close vicinity to the slit diaphragm as shown by anti-Flag labeling (FIG. 9D). All together, these data show that overly active TRPC6 or increased amounts of TRPC6 can be sufficiently neutralized in vivo by neutralizing the amounts of expressed TRPC6. Reduction of TRPC6 expression in injured podocytes is accompanied with increased synaptopodin expression and a reduction of proteinuria.

Discussion

The findings presented herein, explain how TRPC6 mediated Ca²⁺ influx regulates the podocyte foot process microfilament system and thus the glomerular filter. In particular, a TRPC6-synaptopodin signaling loop uses Ca⁺ sensitive serine/threonine enzymes such as PKA and calcineurin to regulate susceptibility of synaptopodin towards the protease cathepsin L and thus affects podocyte RhoA function. Under pathological conditions such as glomerular injury, an increase in wt TRPC6 expression leads to increased cellular Ca²⁺, activation of calcineurin, dephosphorylation of synaptopodin, disrupted interaction of synaptopodin with 14-3-3 proteins that allow the presentation of cathepsin L cleavage sites with subsequent synaptopodin degradation, foot process effacement and proteinuria. This signaling cascade suits well to explain some of the deleterious effects that have been observed in conditions with overly active TRPC6 such as in FSGS-causing TRPC6 gene mutations or after induction of wt TRPC6 channel expression in acquired forms of glomerular injury such as in Membranous Nephropathy. Maintenance of physiological TRPC6 activity can be achieved by either reducing the levels of expressed TRPC6 in the podocyte or by diminishing the amounts of Ca²⁺ influx channeled by TRPC6 using a podocyte-specific TRPC6 pore mutant (FIGS. 9D, 9E). It is important to note that TRPC6 mediated Ca²⁺ flux has a general small capacity but is a sufficiently strong mediator in subcellular compartments such as podocyte foot process and neuronal growth cones. In fact, other TRPC subunits such as TRPC5 have been shown to be closely involved in the regulation of growth cone motility. TRPC6 is shown as regulator of podocyte motility. The absence of TRPC6 leads to a reduced motility of podocytes whereas injured podocytes generally exhibit an increased cellular motility. It is conceivable that the downregulation of TRPC6 by siRNA or the expression of the podocyte-specific pore mutant exert these effects through stabilization of synaptopodin, steady Rho A levels and diminished activity of cdc42 and rac-1 levels. The rapid recovery of mice perfused with polycations such as protamine sulfate followed by the anion heparin supports the concept of a highly dynamic podocyte foot process system.

While the data herein identify synaptopodin as one downstream target of TRPC6 that can reciprocally feed back to TRPC6 channel localization and function, other targets need to be identified. The phenotype of the TRPC6 knockout mice that have normal foot processes would also be better defined. However, these mice have likely a compensation of other TRPC subunits that can compensate largely for TRPC6 function at least during young age. It is possible that TRPC6 knockout mice require a pathological challenge for example with LPS or protamine sulfate before a reduced cytoskelatal plasticity might become obvious.

The discovery of TRPC6 function in podocyte continues to be an exciting area of glomerular research that directly or through its downstream effector has direct implications for novel therapeutic development.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A method of measuring or quantifying calcineurin activity in vitro or in vivo comprising:
transforming a cell with a reporter construct, wherein the reporter construct comprising a polynucleotide encoding a nuclear factor of activated T cells (NFAT); a selectable marker and a reporter gene;
measuring NFAT activity in the sample;
correlating NFAT activity to calcineurin activity; and;
measuring or quantifying calcineurin activity.

2. The method of claim 1, wherein the reporter gene is operably linked to the NFAT polynucleotide comprising an NFAT response element.

3. The method of claim 2, wherein the reporter gene is under control of the NFAT response element.

4. The method of claim 1, wherein NFAT phosphorylation or dephosphorylation state is a measure of NFAT activity.

5. The method of claim 1, wherein NFAT is localized in different intra-cellular compartments depending on NFAT phosphorylation states.

6. The method of claim 1, wherein phosphorylated NFAT is detected in podocyte cytoplasm and dephosphorylated NFAT is detected in podocyte nuclei.

7. The method of claim 1, wherein NFAT activity is detected by a reporter assay.

8. The method of claim 2, wherein the reporter gene is a luciferase gene or mutants thereof.

9. A reporter construct comprising: polynucleotide encoding a nuclear factor of activated T cells (NFAT), fragments or mutants thereof; a selectable marker and a reporter gene.

10. The reporter construct of claim 9, wherein the reporter gene comprising at least one wild-type luciferase, luciferase mutants or combinations thereof.

11. The reporter construct of claim 9, wherein the polynucleotide encoding a nuclear factor of activated cells (NFAT) comprises an NFAT response element.

12. The reporter construct of claim 11, wherein the reporter gene is under control of the NFAT response element

13. An isolated cell comprising the reporter construct of claim 9.

14. A method of diagnosing an immune-related disease or condition in a patient comprising:
obtaining a sample from a patient and measuring NFAT activity in the sample;
correlating NFAT activity to calcineurin activity; and,
diagnosing an immune-related disease or condition in a patient.

15. The method of claim 14, wherein the immune-related disease comprising: acute immune diseases, chronic immune diseases, autoimmune diseases, inflammation, tissue or organ transplant graft rejections or graft-versus-host disease.

16. A method of diagnosing kidney disease or condition in a patient comprising: obtaining a sample from a patient and measuring NFAT activity in the sample;
correlating NFAT activity to calcineurin activity; and,
diagnosing kidney disease or condition in a patient.

17. A method of diagnosing cancer in a patient comprising: obtaining a sample from a patient and measuring NFAT activity in the sample;
correlating NFAT activity to calcineurin activity; and,
diagnosing cancer in a patient.

18. A method of screening and identifying novel therapeutic compounds comprising:
contacting an NFAT molecule or a cell comprising a reporter construct, wherein the reporter construct comprising a polynucleotide encoding a nuclear factor of activated T cells (NFAT), a selectable marker and a reporter gene;
measuring NFAT activity in the presence or absence of the compound;
correlating NFAT activity to calcineurin activity; and;
screening and identifying novel therapeutic compounds.

19. The method of claim 18, wherein the wherein NFAT phosphorylation or dephosphorylation state is a measure of NFAT activity.

20. The method of claim 18, wherein an NFAT molecule comprises an NFAT polynucleotide, oligonucleotide, peptide, polypeptide, mutants, variants, fragments or combinations thereof.

21. The method of claim 18, wherein the method is a high-throughput assay.

22. A method of treating a disease or disorder characterized by proteinuria comprising administering to a patient in need thereof, an effective amount of an agent whereby the agent modulates NFAT activity and/or an agent identified by the method of claim 18.

23. The method of claim 22, wherein the disease or disorder characterized by proteinuria comprising: glomerular diseases, membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis (FSGS), IgA nephropathy, IgM nephropathy, membranoproliferative glomerulonephritis, membranous nephropathy, sarcoidosis, Alport's syndrome, diabetes mellitus, kidney damage due to drugs, Fabry's disease, infections, aminoaciduria, Fanconi syndrome, hypertensive nephrosclerosis, interstitial nephritis, Sickle cell disease, hemoglobinuria, multiple myeloma, myoglobinuria, Wegener's Granulomatosis or Glycogen Storage Disease Type 1.

24. A method of treating an immune-related disease or disorder comprising administering to a patient in need thereof, an effective amount of an agent whereby the agent modulates NFAT activity and/or an agent identified by the method of claim 18.

25. The method of claim 24, wherein an immune-related disease comprises: acute immune diseases, chronic immune diseases, autoimmune diseases, inflammation, tissue or organ transplant graft rejections or graft-versus-host disease.

26. A composition comprising at least one agent wherein the agent modulates NFAT expression, function or activity in vivo.

27. A composition comprising a therapeutically effective amount of at least one agent which modulates expression, function or activity of transient receptor potential cation channels (TRPC) in vivo.

28. The composition of claim 27, wherein the at least one agent decreases expression of one or more TRPCs in vivo as compared to a normal control.

29. A method of treating kidney diseases or disorders in a patient, comprising administering to a patient in need thereof, a therapeutically effective amount of at least one agent which modulates expression, function or activity of at least one TRPC as compared to a normal control, and;
treating a kidney disease or disorder.

30. The method of claim 29, wherein the expression, activity or function of a TRPC is measured by measuring intracellular Ca²⁺ levels, activation of calcineurin, phospphoryltaion or dephosphorylation of synaptopodin, interaction of synaptopodin with 14-3-3 proteins or cathepsin L cleavage.
